(19) United States
(12) Patent Application Publication
(10) Pub. No.: US 2006/0088896 A1
(43) Pub. Date: Apr. 27, 2006

(54) Diagnostic device for rapid determination of buprenorphine

(76) Inventors: Silverio Casolaro, Lucca (IT); Alessia Zampieri, Lucca (IT)

Correspondence Address: NIXON & VANDERHYE, PC, 901 NORTH GLEBE ROAD, 11TH FLOOR, ARLINGTON, VA, 22203, US

(21) Appl. No.: 10/529,582

(22) PCT Filed: Oct. 9, 2003
(86) PCT No.: PCT/EP03/11160

§ 371 Date: Mar. 29, 2005

(30) Foreign Application Priority Data
Oct. 15, 2002 (IT) MI2002A002186

(51) Int. Cl. G01N 33/537; G01N 33/53; G01N 33/543

(52) U.S. Cl. 435/7.92

(57) ABSTRACT

This invention relates to a method of detecting buprenorphine in biological fluids (especially urine), and to a device and kit which use chromatographically mobile reagents labelled with gold clusters. The method involves contacting urine sequentially with anti-buprenorphine antibodies labelled with gold clusters and with buprenorphine immobilised on a porous support, in order to detect the presence of buprenorphine in the sample by means of a competitive reaction. The device according to the invention consists of a rack which can be filled with a number of strips; the kit consists of a box with separate sections for the strips and a separate compartment which holds the rack.

### FIELD OF INVENTION

This invention relates to a device which can easily be used, even by unskilled personnel, to determine the presence of buprenorphine in biological fluids (especially urine) by applying an immunochemical technique.

More particularly, this invention relates to a device consisting of a reusable rack and disposable strips on which the buprenorphine detection test is performed.

The rack, which is the fixed part of the test device, holds a number of strips, so that a multitest can be performed for simultaneous determination of buprenorphine and other drugs of abuse.

The strip rack and test strips can be inserted into a kit consisting of a box with separate sections (one for each type of strip) and a separate compartment designed to hold the multitest rack. This kit is useful not only for transporting the devices needed for rapid tests, but also for their storage.

The invention also relates to a method for rapid determination of buprenorphine in biological fluids such as blood, saliva etc. and especially urine, characterised in that gold cluster conjugates (the preparation of which is described in patent U.S. Pat. No. 5,360,895) are used to display the immunochemical reaction. This technique is very useful in this specific case, because it increases the sensitivity of detection compared with the rapid diagnostic methods currently in use, which are based on colloidal gold. This greater sensitivity is particularly important in the case of buprenorphine, which is more potent than morphine and consequently taken at much lower doses which produce very low and hardy detectable concentrations in the biological fluids.

### STATE OF THE ART

Buprenorphine is an opioid (a synthetic molecule with morphine-like properties) which performs an analgesic action that depresses the central nervous system. Its formula is:

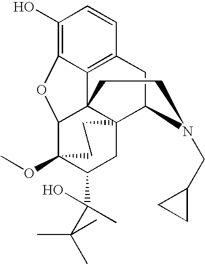

It is used in the treatment of various forms of pain (such as long-term treatment of cancer patients) and has a potent analgesic effect, 25 to 50 times more potent than morphine, with a safer therapeutic index. Intravenous or intramuscular doses of 0.3-0.4 mg of buprenorphine are usually considered equianalgesic to 10 mg of morphine. These values are still under study, so a comparison of the action of the two drugs has not yet been finalised. Buprenorphine is also used in the treatment of opiate addiction because it is a partial agonist of receptor μ1 and an antagonist of receptor κ3, with a high affinity for opiate receptors and slow dissociation from those sites, and a long half-life (48-72 hours). This gives it a longer-lasting analgesic action than morphine. Studies are also being conducted on the use of buprenorphine in the treatment of addiction to other drugs (cocaine and opiates).

The pharmacokinetics of buprenorphine after oral, intramuscular and intravenous administration have been extensively studied. Sublingual administration has the advantage of reducing the damage caused by intravenous drug abuse (exchange of syringes and transmission of disease). Moreover, as a result of its long-lasting action, a therapeutic protocol of doses given on alternative days is possible, with obvious advantages.

The efficacy of buprenorphine is dose-dependent; a dose of 8 mg/day is equivalent to 60 mg of methadone.

However, some studies (“Consumption of buprenorphine and other drugs among heroin addicts under ambulatory treatment”, Addiction, 1993, 88, 1341-9; “Intravenous buprenorphine self-administration by detoxified heroin abusers”, J Pharmacol: Exp. There., 2002, 301, 266-76) have found that buprenorphine is also abused.

Buprenorphine is therefore on a par with other drugs of abuse (morphine, cocaine, heroin, etc.).

A rapid diagnostic test is therefore needed to identify the presence of buprenorphine in the biological fluids.

The diagnostic methods currently in use to detect buprenorphine, are only applicable in specialist laboratories, as they require skilled personnel and expensive instruments, such as HPLC (“Analysis of buprenorphine in urine specimens”, J. Forensic Sci., 1992, 37, 82-9); radioimmunoassay technique (“Development of a radioimmunoassay for the determination of buprenorphine in biological samples”, Analyst, 1993, 118, 137-143); thin-layer chromatography (“Determination of buprenorphine and its N-dealkylated metabolite in urine by TLC densitometry”, Ind. J. Pharmacol., 1994, 26, 288-91); gas chromatography (“Subnanogram-concentration measurement of buprenorphine in human plasma by electron-capture capillary gas chromatography: application to pharmacokinetics of sublingual buprenorphine”, Clin. Chem., 1997, 2292-2302); and mass spectroscopy (“Determination of buprenorphine and norbuprenorphine in urine and hair by gas chromatography-mass spectroscopy”, J. Anal. Toxicol., 1999, 23, 270-9).

### DESCRIPTION OF THE INVENTION

This invention relates to a diagnostic device for the qualitative identification of buprenorphine in a biological fluid which allows a rapid test to be performed on the spot (in an Accident and Emergency Department, by sports organisations, or by public officials such as traffic police officers, prison warders, etc.). The device according to the invention comprises a strip consisting of a porous, preferably microporous, material which is particularly absorbent, such as a cellulose material, on which the antibody-gold cluster conjugate is adsorbed. Said porous support is divided into a first zone on which anti-buprenorphine antibodies labelled with gold clusters have been adsorbed, a second zone on which buprenorphine conjugated with albumin has been immobilised, and a third control zone on which a different antigen-antibody reaction takes place, which is wholly independent of the presence or absence of buprenorphine in the sample to be analysed.

A further aspect of the invention relates to a method for the qualitative determination of buprenorphine in a biological fluid, which involves contacting the biological fluid sequentially with anti-buprenorphine antibodies labelled with gold clusters which are reversibly adsorbed on a porous support for detection of the immunocomplex by competition with buprenorphine immobilised in a reading zone of said porous support.

The use of antibodies labelled with gold clusters offers greater sensitivity than the antibodies labelled with colloidal gold which have been conventionally used in rapid diagnostic methods to date.

The invention also includes a kit consisting of a rack and a number of diagnostic devices in the form of disposable porous (preferably microporous) strips.

Finally, a further aspect of the invention relates to a kit of low weight, designed to store and easily transport the reagents contained in it. Said kit consists of a transparent box, in which the rack and the porous strips are housed in separate compartments.

The immunochemical determination of buprenorphine according to the invention uses the capillarity of the porous material, which acts as a vehicle; anti-buprenorphine antibodies prepared by known methods, for example by first reacting the buprenorphine with bovine albumin (according to the carbodiimide method) to induce the production of antibodies, are adsorbed on a suitable area of said material.

The anti-buprenorphine antibodies thus obtained are labelled with gold clusters. A cluster is a coordination complex containing a nucleus of gold atoms (in a specific number) which are geometrically well delineated and have an organic coating. This coating enables the cluster to bind to the necessary antibodies with a covalent bond so that the resulting gold/antibody complex is a highly stable molecule, unlike the complexes obtained with colloidal gold particles. Colloidal gold particles present a number of drawbacks: as they are not chemically bound to the antibodies (which are simply adsorbed onto their surface), the stoichiometry of the bond cannot be controlled. The antibodies can therefore dissociate from the complex, leading to weaker signals. Moreover, these particles are negatively charged, which means that they can bind non-specifically to other molecules, thus giving false negatives. Gold clusters thus offer numerous advantages, because they are not charged and are smaller than colloidal gold particles, which results in greater sensitivity, as the gold/antibody ratio is increased.

Once the antibodies have been coupled to the gold cluster, the complex is impregnated and dried on one end of the porous support of the device. Buprenorphine, preferably conjugated with a protein such as BSA (bovine serum albumin), is immobilised in a detection area of the support.

The test reaction is preferably the competitive type. When the biological fluid comes into contact with the zone containing the antibody/gold cluster conjugate, if said fluid contains the drug in question the drug will react with the antibody/gold cluster conjugate, inhibiting the reaction with the buprenorphine immobilised on the detection site of the porous strip. The absence of a coloured line indicates a positive test (presence of buprenorphine in the biological fluid). If the drug is not present in the biological fluid, the antibody/gold cluster conjugate will react with the immobilised buprenorphine to produce a coloured line (negative test).

A control line with a different antigen-antibody reaction is also prepared on the strip so that it is not influenced by the presence or absence of buprenorphine in the test fluid.

In conclusion, when the test has been performed, if a control line appears on the strip and no line in the detection zone, the test result is positive (buprenorphine above the threshold value is present); if two lines appear, in the control and detection zones, the test is negative; if no line is visible, or only one line in the detection zone, the test is invalid and must be repeated.

The strip which performs the diagnostic test for buprenorphine can be associated with other strips for simultaneous determination of a number of drugs of abuse, using a multiple rack containing several strips. This rack can be rectangular in shape with transparent walls and two openings, at the top and bottom, with channels in the rack into which the test strips are slotted.

Instead of being fixed and therefore pre-packaged in a pre-determined way, the strips can therefore be slotted in when the test is performed, only strips which detect the drugs of interest being inserted.

The rack can thus be filled with a single strip or with two, three, four or five strips, and so on, on a single occasion.

The test will consequently be cheaper if it is specifically targeted on a number of drugs of abuse.

Moreover, the strip rack can be re-used by replacing the used strips with new strips for a subsequent test.

### DESCRIPTION OF DRAWINGS

FIG. 1 Plan view of the rack in the closed configuration.

FIG. 2 Plan view of the rack in the configuration open on two sides, top and bottom.

FIG. 3 Cross-section of fixed part 2 illustrated in FIG. 1 and FIG. 2.

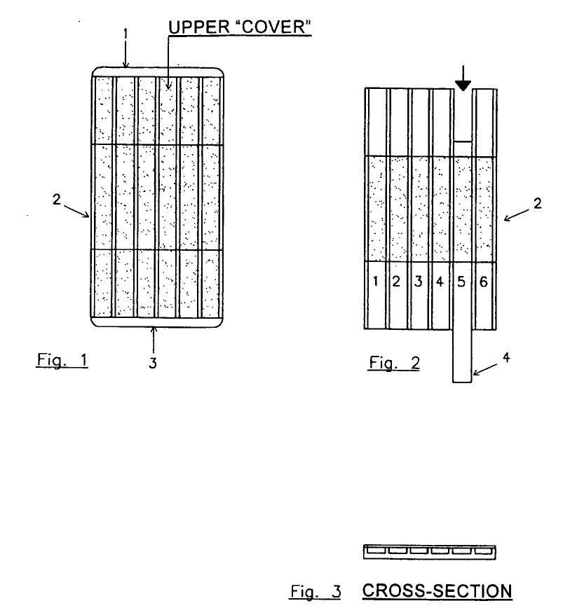

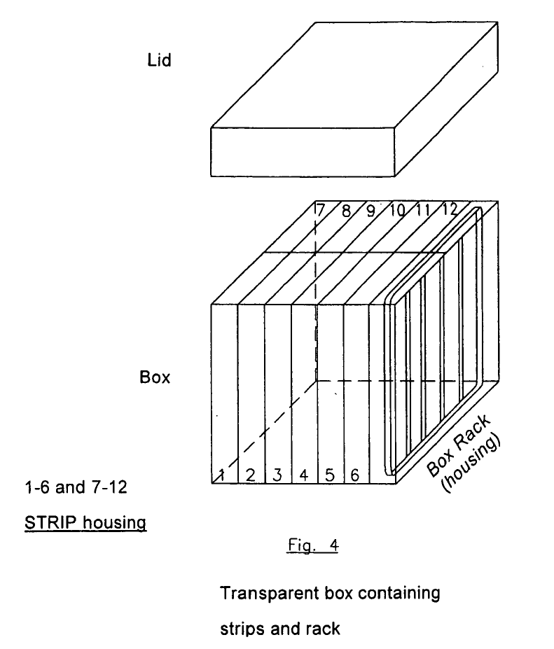

As will be seen in FIG. 1, the drug detection test rack consists of a fixed part 2 and two removable parts 1 and 3.

FIG. 2 shows fixed part 2 and strips 4 inserted in the rack. These strips project into the top and bottom parts.

The strips, which are slotted into the channels, are glued to supports made of plastic or another material, so as to isolate them from the fixed part of the rack.

The strips are slotted in from above and secured by lateral or vertical thickenings just before they exit from the fixed part. When the test has been performed, the strips are pushed downwards and released from the fixed part, thus separating from the rack.

The fixed part of the device can consequently be re-used, thus saving not only the rack, which is re-used, but also the strips, if the tests are specifically targeted and therefore performed in a limited number.

As will be seen from FIG. 4, the rapid drug testing strips and the rack, which is used to support the strips and perform a simultaneous “multitest”, are slotted into a kit constituted by a transparent plastic box which is used for the purpose of storage and transport.

As the box is transparent, the test for which each strip is designed can be read from the outside, so that the strip can easily be located and removed.

1. Diagnostic device for the determination of buprenorphine in a biological fluid, comprising a porous support divided into a first zone on which anti-buprenorphine antibodies labelled with gold clusters have been adsorbed, a second zone on which buprenorphine has been immobilised, and a third zone on which IMMUNOREACTIVE substances that give a different antigen-antibody reaction, independently of the presence of the drug in the sample to be analysed, are adsorbed.

2. Device as claimed in claim 1, wherein the immobilised buprenorphine is conjugated with an immunogenic protein.

3. Device as claimed in claim 2, wherein the immobilised buprenorphine is conjugated with albumin.

4. Device as claimed in claim 1, wherein the porous support is constituted by cellulose.

5. Device as claimed in claim 4, in the form of strip of microporous paper.

6. A rack comprising a plurality of devices as claimed in claim 1, and optionally other devices for the determination of other drugs of abuse.

7. A rack as claimed in claim 6, wherein the strips are separated from one another in compartments open at each end from which they can be removed, said compartments being formed in a re-usable rack closed by two removable lids.

8. A transparent box constituting the kit, comprising a plurality of the devices claimed in claim 1, the rack and optionally other devices for the determination of other drugs of abuse.

9. A method for the determination of buprenorphine in biological fluids which involves contacting the biological fluid sequentially with anti-buprenorphine antibodies labelled with gold clusters, reversibly adsorbed on a porous support, and detecting the immunocomplex by competition with buprenorphine immobilised in a reading zone of said porous support.
